(19) United States
(12) Patent Application Publication
(10) Pub. No.: US 2004/0192579 A1
(43) Pub. Date: Sep. 30, 2004

(54) Compositions comprising matrine and dictamnine for treating or preventing cancer and other diseases

(76) Inventors: John Wah Tze, Vancouver (CA); Theresa Chiang Tze, Vancouver (CA); Peizhong Lin, Beijing (CN); Stephen Lam, Vancouver (CA); Joseph Tai, Vancouver (CA); Frisi Hobbe Smit, Utrecht (NL); Adrianus Lambertus Bertholdus Van Helvoort, Wageningen (NL); Robert Johan Joseph Hageman, Wageningen (NL)

Correspondence Address: YOUNG & THOMPSON, 745 SOUTH 23RD STREET 2ND FLOOR, ARLINGTON, VA, 22202

(21) Appl. No.: 10/468,892

(22) PCT Filed: Feb. 26, 2002
(86) PCT No.: PCT/EP02/02351

Related U.S. Application Data
(63) Continuation of application No. 09/793,251, filed on Feb. 26, 2001, now abandoned.

(51) Int. Cl. A61K 35/78; A61K 38/17

(52) U.S. Cl. 514/2

(57) ABSTRACT

The present invention is concerned with the treatment or prevention of cancer, H. pylori infections, chronic inflammatory conditions, cardiovascular diseases or cerebral vascular diseases in a mammal, comprising the administration of the mammal of an effective amount of a composition containing a matrine component and dictamnine. Another aspect of the invention relates to a composition comprising at least 15 μmole of a matrine component and at least 0.15 μmole of dictamnine.

### TECHNICAL FIELD OF THE INVENTION

The present invention is concerned with a method of treating or preventing cancer in a mammal, said method comprising the administration of a combination of active principles that can be found in certain herbs.

The invention also relates to a method of treating or preventing, H. pylori infections, chronic inflammatory conditions, cardiovascular diseases or cerebral vascular diseases in a mammal, said method comprising the administration of the same combination of active principles.

Another aspect of the invention relates to a composition comprising this combination of active principles.

### BACKGROUND OF THE INVENTION

Nowadays a huge number of different drugs is available for combating a large variety of cancers. Most of these drugs are composed of individual or combinations of chemicals that do not occur in nature. While some of these chemicals are effective, many have serious side effects which prevent their long-term and/or recurrent use.

Traditionally, the focus of cancer research has been in developing therapies and treatment for patients already afflicted with cancer. However, over the last few decades, new insights in the development of cancer as a disease have been gained. It is now understood that cancer is not the result of a single initiation event, but of a gradual, multi-step process characterised by a period of several years between the initiation event and the onset of invasive or metastatic disease. In general, the process of carcinogenesis can be divided into three phases: initiation, promotion and progression. In initiation, a fixed genetic mutation results from the interaction of a carcinogen with DNA. The extent of the molecular change depends on a number of factors including the nature of the carcinogen, the rate and type of carcinogenic metabolism and the response of the DNA repair system. The next phase, promotion, may occur over extended periods of time and is characterised by the proliferation of the altered cells. This phase may be affected by agents that alter growth rates. During the final phase, progression, genetic and phenotypic changes occur which ultimately cause the development of premalignant lesions into invasive cancer.

The multi-step nature of carcinogenesis suggests the possibility of intervention at a precancerous state. This is the basis of chemoprevention, which refers to the use of natural or synthetic agents to prevent the development of cancer, either by blocking the DNA damage that initiates the carcinogenesis process or by arresting/regressing existing pre-malignant lesions.

Since the mid-1950's, research has been directed at finding components with potential chemopreventive properties. The search for these agents has demonstrated an unique challenge. Chemopreventive agents must have low toxicity and be relatively free of side effects because they are intended for administration to healthy people over long periods of time. This is in direct contrast with chemotherapy drugs. Chemotherapeutic drugs are chosen for their ability to kill tumor cells, but because they are also toxic to healthy cells, they usually cause harmful side effects.

The best example to-date that chemoprevention can prevent cancer is Tamoxifen. Tamoxifen is an estrogen antagonist. In women at high risk of breast cancer, the Breast Cancer Prevent Trial showed a 49% decrease in invasive breast cancer and a 50% decrease in non-invasive breast cancer with Tamoxifen versus placebo (J Natl Cancer Inst 1998; 90: 1371-88).

One of the major sources of potential chemopreventive agents is plants. For example, consumption of cruciferous vegetables, such as broccoli, cauliflower and cabbage is associated with a lower risk of various cancers. Fruits and vegetables contain a number of potentially active chemopreventive compounds, such carotenoids, dithiolthiones and isothiocyanates. These compounds have been shown to be capable of inhibiting the development of tumours of the lungs, colon, mammary glands and bladder in laboratory animals.

A variety of Chinese herbs have been used for centuries to treat different diseases. Although some of these herbs have been used to treat patients with cancer, they are not considered to be disease specific. Herbs such as Sophora tonkinensis, Polygonum bistorta, Prunella vulgaris, Sonchus brachyotus, Dictamnus dasycarpus Turcz and Dioscorea bulbifera comprise varying amounts of components that have been shown to display anti-proliferative effects in certain in vitro assays. A herbal composition containing one or more of the aforementioned herbs is known as ZSP or Zeng Sheng Ping; however, the exact formulation of the composition is not known. In traditional Chinese herbal medicine, it is common practice to substitute selected herbs with other herbs and to vary the relative amounts of the individual herbs, depending upon the symptoms of an individual patient and/or local availability of certain herbs.

From the above it will be evident that there is a requirement for effective chemopreventive agents that do not give rise to harmful side-effects and which are consistently effective against a wide variety of cancers.

### SUMMARY OF THE INVENTION

The inventors have found that the aforementioned requirement is met by a composition that comprises a special combination of active principles that can be obtained from different herbs that have been in use in herbal medicine for a very long time. The composition according to the invention may advantageously be employed to both prevent and treat various forms of cancer and does not give rise to harmful side effects. Surprisingly it was found that the present composition is not only effective in the treatment of cancer, but also that it can be used to treat or prevent Helicobacter pylori infections, chronic inflammatory conditions, cardiovascular diseases and cerebral vascular diseases in mammals.

More particularly the inventors have unexpectedly discovered that the combined administration to a mammal of a matrine component and dictamnine in an effective amount, may suitably be used to treat or prevent cancer as well as to treat or prevent pathogenic infections of the gastrointestinal tract.

The inventors have further discovered that the additional inclusion of atriterpenoid component and/or aurapten may further improve the chemopreventive and therapeutic properties of the present composition. Examples of triterpenoid components that may suitably be employed include sapogenins (e.g. diosgenin) and limonoids (e.g. obacunone and fraxinellone derivatives and isomers).

It has been demonstrated in an in vitro assay that the addition of 0.1 mg/ml matrine can significantly inhibit cell-growth of K-562 cells (Zhang et al., “Effects of Matrine on proliferation and differentiation in K-562 cells”; Leuk Res. 2001 Sep; 25(9):793-800).

Matrine and oxymatrin have been mentioned as active components of Sophora by Leung and Dharmananda in Internet publications. Reference is made therein to practices in Chinese medicine wherein Sophora flavescens and Sophora subprostata are used in cancer therapy. The recommended oral doses are in the range of 300-600 mg matrine per day.

Moalic et al. (“A plant steroid, diosgenin, induces apoptosis, cell cycle arrest and COX activity in osteosarcoma cells”, FEBS Letters 506 (2001) 225-230) have investigated the effect of diosgenin on the proliferation rate, cell cycle distribution and apoptosis in the human osteosarcoma 1547 cell line. Diosgenin treatment was found to cause an inhibition of 1547 cell growth with a cycle arrest in G₁ phase and apoptosis induction.

Limonoids have been found to have anti-carcinogenic activity in laboratory animals. The furan moiety attached to the D-ring is specifically responsible for detoxifying of the chemical carcinogen by induction of the liver glutathione-S-transferase enzyme system (Lam, et al., 1994, Food Technol. 48:104-108).

### DETAILED DESCRIPTION OF THE INVENTION

One aspect of the invention relates to a method of treating or preventing cancer or pathogenic infections of the gastointestinal tract in a mammal, said method comprising the administration to the mammal of an effective amount of a composition containing a matrine component and dictamnine.

The term “cancer” refers to a wide variety of diseases in which cells of different origin proliferate in an uncontrolled and relatively rapid way.

The term “matrine component” as used herein includes matrine, oxymatrine and precursors capable of liberating either of these components iii vivo when used in accordance with the present invention. The term “dictamnine” refers to dictamnine (4-methoxyfuro(2,3-b)-quinoline as well as precursors thereof. It is furthermore noted that whenever reference is made to matrine, oxymatrine, dictamnine or other active principles specifically named herein, the salts of these substances are also encompassed.

The method of the invention is particularly effective when used to treat or prevent a cancer selected from the group consisting of esophageal cancer, stomach cancer, colon cancer, lung cancer, prostate cancer, bladder cancer, liver cancer, breast cancer, cervical cancer, ovary cancer, lymfoma's, melanomia and leukemia.

Another aspect of the invention relates to a method of treating or preventing Helicobacter pylori infections in a mammal, said method comprising the administration to the mammal of an effective amount of a composition containing a matrine component and dictamnine.

Yet another aspect of the invention relates to a method of treating or preventing chronic inflammatory conditions in a mammal, said method comprising the administration to the mammal of an effective amount of a composition containing a matrine component and dictamnine.

Yet another aspect of the invention relates to a method of treating or preventing cardiovascular diseases in a mammal, said method comprising the administration to the mammal of an effective amount of a composition containing a matrine component and dictamnine.

Yet another aspect of the invention relates to a method of treating or preventing cerebral vascular diseases in a mammal, said method comprising the administration to the mammal of an effective amount of a composition containing a matrine component and dictamnine.

In a preferred embodiment of the present methods, the matrine component is administered in an average daily amount of at least 0.2 μmole per kg of bodyweight, preferably of at least 0.5 μmole per kg of bodyweight. The average daily amount of the matrine component that is administered in accordance with the present method preferably remains below 17 μmole per kg of bodyweight, more preferably below 10 μmole per kg of bodyweight. The chronic administration of the matrine component in daily amounts of more than 17 μmole per kg of bodyweight does not offer much advantage in terms of efficacy, but does have the drawback that it may cause undesirable side effects such as dizziness and stomach upset.

Good results can be obtained, particularly in mammals with a bodyweight in excess of 50 kg, with the present methods if the matrine component is administered in an amount which is equivalent to a daily oral dosage of at least 7 mg matrine, preferably at least 9 mg matrine. Usually the administered amount does not exceed the equivalent of a daily oral dosage of 200 mg matrine, preferably it does not exceed the equivalent of a daily oral dosage of 100 mg matrine.

The matrine components used in the present methods and composition are preferably selected from the group consisting of matrine (sophocarpidine), oxymatrine and mixtures thereof. Matrine and oxymatrine are alkaloids that are found in plants of the Sophora species. In one embodiment of the invention the composition comprises plant material derived from a plant belonging to the Sophora species, preferably said plant material is derived from Sophora flavescens or Sophora subprostata. The roots of plants of the Sophora species are preferred as a source of matrine components.

The inventors have unexpectedly found that if plants belonging to the Sophora species are combined with certain herb varieties to produce an aqueous herbal extract, the recovery of matrine in the resulting extract is very low. In addition it was discovered that much higher recoveries can be obtained if the plant material derived from a Sophora species is subjected to an aqueous extraction step in the absence of any of these interfering herb varieties. Examples of herb varieties that may negatively influence the matrine recovery include plants belonging to the Dictamnus species.

In the present methods dictamnine is usually administered in an average daily amount of at least 2 nanomole (about 0.4 μg) per kg of bodyweight, preferably of at least 4 nanomole per kg of bodyweight. The average daily amount of the dictamnine that is administered in accordance with the present methods preferably remains below 15 nanomole per kg of bodyweight, more preferably below 10 nanomole per kg of bodyweight. The application of dictamnine in amounts that exceed an average daily dosage of 15 nanomole per kg of bodyweight is not desirable because such high dosages do not provide a significant advantage in terms of efficacy and because high doses of this particular substance might produce undesirable side effects.

The dictamnine employed in the present methods may suitably be obtained from plant sources such as plants belonging to the species Dictamnus. Hence, in a preferred embodiment, the present composition comprises plant material derived from a plant belonging to the species Dictamnus, more preferably the plant material is derived from Dictamnus dasycarpus (Turcz).

Because dictamnine is hardly soluble in water, said component is suitably included in the present composition in the form of dried and optionally ground plant material or alternatively in the from of a non-aqueous (apolar) extract. Because plants from the species Dictamus contain a number of water-soluble components (e.g. aurapten, limonin, obacunone, sterols) that are believed to contribute to the chemopreventive and therapeutic properties of the present composition, it is preferred to also include an aqueous extract of a plant material obtained from the species Dictamus.

Particularly good results can be obtained with the present methods if, in addition to the administration of the matrine component and dictamnine, it comprises the co-administration of a triterpenoid component selected from the group consisting of sapogenins, limonoids, triterpenoids with a oleanane skeleton, triterpenoids with a lupane skeleton, precursors of these triterpenoid components and mixtures thereof, in an average daily amount of at least 40 nanomole per kg of bodyweight, preferably of 80-4800 nanomole per kg of bodyweight. Precursors of triterpenoids are substances that are capable of liberating the aforementioned triterpenoids in vivo when used in accordance with the present invention. Saponosides, glycosides and aglycones of sapogenins are good examples of precursors of sapogenins. Similarly glucosides and aglycones of limonoids can suitably be employed as precursors of limonoids.

The triterpenoid components used in accordance with the invention typically have a tetra- or pentacyclic planar structure. Typical examples of triterpenoid components include saikosaponins, certain soy saponins, triterpenoids from gleditsia, centella, taraxacum, vascum or platycodon. The triterpenoid component is preferably selected from the group consisting of sapogenins, limonoids, triterpenoids with an oleanane skeleton, triterpenoids with a lupane skeleton and precursors of these triterpenoid components and mixtures thereof.

Sapogenins are compounds resulting from the acid hydrolysis of saponosides, which are heterosides of very high molecular weight which are found in some plant varieties. Limonoids are a group of chemically related triterpene derivatives found in the Rutaceae and Meliaceae families. Limonoids are among the bitter principles found in citrus fruits such as lemons, lime, orange and grapefruit. They are also present as glucose derivatives in mature fruit tissues and seed, and are one of the major secondary metabolites present in Citrus.

In a preferred embodiment of the invention, the sapogenins are selected from the group consisting of diosgenin, hecogenin, smilagenin, sarsapogenin, tigogenin, yamogenin, yuccagenin, glycosides of said sapogenins, aglycones of said sapogenins and mixtures thereof. Suitable sources for these sapogenins include fenugreek species and Mexican wild yam. Some plant isolates that contain high levels of sapogenins also contain significant concentrations of diosbulbine. Diosbulbine is believed to cause undesirable side-effects and consequently the present method preferably does not comprise the administration of appreciable amounts of diosbulbine. Preferably the daily amount of diosbulbine administered in the present method does not exceed 0.1 mg, more preferably it is below 10 μg.

As regards the limonoids it is preferred to employ a limonoid selected from the group consisting of limonin, nomilin, 12-hydroxy-amdorastatin, obacunone, 7-alpha-acetylobacunol, fraxinellone, dasvcarpol, calodendrolide, 7-alpha-acetyldihydronomilin, limonin diosphenol, rutaevin, isofraxinellone, 6-hydroxy fraxinellone, precursors of these limonoids and mixtures thereof. Most preferably the limonoid is selected from the group consisting of limonin, fraxinellone, obacunone, glycosides of said limonoids, aglycones of said limonoids and mixtures thereof. In the present composition the limonoids need not be protected through the incorporation of tocopherols or tocotrienols as described in U.S. Pat. No. 6,251,400.

Examples of triterpenoids with an oleanane skeleton that can advantageously be employed in the present method include oleanolic acid and ursolic acid. An good example of a triterpenoid with a lupane skeleton is burlinic acid.

It was found that particularly good results can be obtained if the present methods further comprises the co-administration of aurapten. The substance aurapten is a representative of the family of coumarins and is suitably obtained from plants belonging to the Citrus or Dictamnus species. In case of plants belonging to the Citrus species, the peels and seeds are a particularly good source of aurapten. The roots of plants belonging to the Dictamnus species, also provide a good source of aurapten. The average daily amount of aurapten that is administered in accordance with the invention usually is at least 5 μg per kg of bodyweight, preferably at least 10 μg per kg of bodyweight. Normally the administered average daily amount of aurapten does not exceed 20 mg per kg of bodyweight. Preferably said amount does not exceed 10 mg per kg of bodyweight, more preferably it is at most 2 mg per kg of bodyweight.

Other plant components that may suitable be co-administered with the matrine component and dictamnine include sterols and flavonoids. Of the plant sterols β-sitosterol and stigmasterol as particularly suited. Kaempferol is the preferred flavonoid.

The present method may suitably employ various modes of administration. Particularly preferred modes of administration include enteral and intravenous administration. Particularly preferred are oral and rectal administration. Most preferably the present method comprises oral administration of the composition, preferably at least once daily oral administration. The active principles used in accordance may be administered separately or combined in a single unit dosage form. Preferably, however, the active principles are administered together in a single unit dosage form.

The active principles in the present composition are preferably administered gradually during the day, e.g. by administering at least 2, or even 3 or more doses per day. Alternatively the present composition is administered as a sustained release formulation that slowly releases the active principles, thus enabling the maintenance of a substantially constant plasma level of the active principles.

Another aspect of the invention relates to a composition comprising at least 15 μmole of a matrine component and at least 0.15 μmole of dictamnine. Preferably the amount of matrine component within the present composition is at least 35 μmole. The amount of matrine component within the composition is preferably below 500 μmole, more preferably below 200 μmole. The aforementioned amounts correspond to the preferred daily doses for human and may be provided in the form of 1-3 unit dosage forms to be administered within about 24 hours. The amount of dictamnine in the composition is preferred to be at least 0.3 μmole. The preferred maximum amount of the dictamnine in the composition is 1.5 μmole. More preferably the maximum amount is 1.0 μmole.

Suitable examples of composition encompassed by the present invention are nutritional and pharmaceutical formulations. The present composition may suitably take the form of a pharmaceutical unit dosage form, e.g. a tablet, a lozenge, an elixir, a liquid, a powder or a suppository. Furthermore the composition may be in the shape of a food product such as a bar, drink, pudding, soup, cookie, spread or ice cream.

The present composition, as mentioned herein before, may advantageously contain a triterpenoid component. Preferably, the amount of triterpenoid component in the composition is at least 2.8 μmole, more preferably it is at least 5.6 μmole The preferred maximum amount of the triterpenoid component in the composition is 4.8 millimole. More preferably the maximum amount is 1 millimole.

In another preferred embodiment, the present composition additionally comprising at least 2 mg of aurapten more preferably at least 4 mg of aurapten. The amount of aurapten in the present composition is preferably kept below 1000 mg. More preferably the maximum amount of aurapten is 200 mg. The amount of diosbulbine contained in the present composition is preferably in the range of 0-0.1 mg, more preferably it is below 0.01 mg.

The remainder of the compositions according to the invention may suitably comprise a large amount of carrier material. Preferably the remainder of the present composition largely consists of edible materials and/or pharmaceutically acceptable excipient.

The present invention encompasses the use of matrine components, dictamnine, triterpenoid component, aurapten, plant sterols and flavonoids that have been derived from plant material as well as synthetic equivalents of these components, i.e. substances that have been obtained from chemical synthesis. The aforementioned active principles can be obtained from plant sources in the form of isolates such as extracts, juices or pressed oils. Alternatively the active principles may be used in the form of comminuted, preferably dried, particles.

In a particularly preferred embodiment of the present method, the administered composition comprises the matrine component and dictamnine in synthetic form. If present, preferably also the triterpenoid component and/or aurapten are employed in synthetic form. The use of these substances in synthetic form offers the advantage that it avoids the incorporation in the present composition of “suspect” components that may occur in plant material together with the desired active principles (e.g. diosbulbine). Because these synthetic substances are chemically identical to their natural counterparts, the advantages associated with the use of these natural chemopreventive and therapeutic agents apply equally to these synthetic substances.

The invention is further illustrated by means of the following examples:

### EXAMPLES

### Example 1

A composition according to the invention was prepared as follows: Dictamnus dasycarpus Turcz (105 g) is ground into a powder form and set aside for later use. Sophora tonkinensis (420 g) is added to ten times its volume in water (approximately 5 L) and then set to boil and decoct for 1.5 hours. The fluid is then removed and saved and another ten times volume of water is added. The mixture is boiled and decocted for another 1.5 hours. The fluid is again removed and then combined with the first portion. The solution is filtered to remove large particles and then concentrated by heating at reduced pressure to S.G. of 1.30-1.35 (at 50° C.). The resulting cream paste is dehydrated at a temperature under 60° C., pulverised and the Sophora tonkinensis powder is then set aside for later use.

Another portion of Dictamnus dasycarpus Turcz (105 g) is mixed with Polygonum bistorta (420 g), Prunella vulgaris (420 g), Sonchus brachyotus (420 g) and Dioscorea bulbifera (100 g). The mixture is added to ten times its volume in water (approximately 15 L), boiled and then decocted for 2 hours. The fluid is then removed and saved. Another ten times volume of water is added and the boiling and decocting process is repeated. The fluid is again removed and added to the first portion. The solution is filtered to remove large particles and then concentrated at reduced pressure to an S.G. of 1.30-1.35 (50° C.) (clear cream paste). The cream paste is then mixed with the Dictamnus dasycarpus powder and the Sophora tonkinensis powder. The mixture is then dehydrated at a temperature below 80° C. The dried herbal extract is then ground to a powder.

An appropriate amount of starch and a small amount of ethanol is added as binding material and 0.3 g tablets are formed. Sugar is then used to coat the tablets. The recipe produces about 1,000 tablets.

Thus one example of the present invention is a combination of Sophora tonkinensis, Polygonum bistorta, Prunella vulgaris, Sonchus brachyotus, Dictamnus dasycarpus Turcz, and Dioscorea bulbiferain an amount of about 21%, 21%, 21%, 21%, 10.5%, and 5.5%, respectively.

### Example 2

In a further embodiment of the present invention Herb F is reduced by about 75%, because certain patients taking the herbal composition of Example 1, showed elevated liver enzymes. Thus, according to this embodiment of the present invention, the formulation comprises a lower concentration of the Herb F. In one example of this embodiment there is provided a composition comprising a combination of Sophora tonkinensis, Polygonum bistorta, Prunella vulgaris, Sonchus brachyotus, Dictamnus dasycarpus Turcz, and Dioscorea bulbifera in an amount of about 21.9%, 21.9%, 21.9%, 21.9%, 11.0%, 1.4%.

### Example 3

In yet a further embodiment of the present invention the Herb F is eliminated entirely from the composition. Thus, according to this embodiment the composition comprises a combination of Herb A, Herb B, Herb C, Herb D and Herb E in an amount of from 6%-38%, 6%-38%, 6%-38%, 6%-38%, and 3%-19%, respectively. In one specific example of this embodiment there is provided a composition comprising a combination of Sophora tonkinensis, Polygonum bistorta, Prunella vulgaris, Sonchus brachyotus and Dictamnus dasycarpus Turcz in an amount of about 22%, 22%, 22%, 22%, and 11 %, respectively.

### Example 4

Solutions A, B and C are prepared from the following compositions:

| | Solution A | Solution B | Solution C |
|---|---|---|---|
| Matrine | 10 mg/l | | 10 mg/l |
| Oxymart | 10 mg/l | | 10 mg/l |
| Dictamnine | | 1 μg/l | 1 μg/l |
| Matrix solution | remainder | remainder | remainder |

An in vitro study is conducted to assess the potential efficacy of these solutions in cancer therapy. Results show that solution C is more effective in preventing and suppressing cell proliferation than either solution A or B.

### Example 5

Solutions D, E and F are formulated on the basis of the following compositions

| | Solution D | Solution E | Solution F |
|---|---|---|---|
| Matrine | 10 mg/l | 10 mg/l | 10 mg/l |
| Oxymart | 10 mg/l | 10 mg/l | 10 mg/l |
| Dictamnine | 2 μg/l | 2 μg/l | 2 μg/l |
| Diosgenin | 0.1 mg/l | | 0.1 mg/l |
| Aurapten | | 0.2 mg/l | 0.2 mng/l |
| Matrix solution | remainder | remainder | remainder |

An in vitro study is conducted to assess the efficacy of the of these solutions in cancer therapy. Results show that solution F is more effective in preventing and suppressing cell proliferation than either solution D or E.

### Example 6

A tablet (0.6 g) was prepared from the following components:

| | |
|---|---|
| Synthetic matrine | 12 mg |
| Ground dictamnus dasycarpus root bark | 100 mg |
| Dry lemon peel | 100 mg |
| Tablet excipients | remainder |

### Example 7

A muesli bar comprising 8 segments of each 5 grams was prepared, each segment having the following composition:

| | |
|---|---|
| 400 mg | of dried aqueous extract of Sophora tokinensis |
| | (providing 15 mg matrine) |
| 100 mg | of ground Dictamus dasycarpus root bark |
| 200 mg | dried orange peels (providing 0.2 mg limonin) |
| 3 g | carbohydrates |
| 1.1 g | fruit and flavours |
| 0.2 g | water |

1-19. (canceled)

20. A method of treating or preventing cancer in a mammal, said method comprising administering to the mammal a composition containing a matrine component and dictamnine, in an amount effective to deliver an average daily amount of at least 0.2 μmole matrine component per kg of bodyweight and an average daily amount of at least 2 nanomole dictamnine per kg of bodyweight.

21. Method according to claim 20, wherein the cancer is selected from the group consisting of esophageal cancer, stomach cancer, colon cancer, lung cancer, prostate cancer, bladder cancer, liver cancer, breast cancer, cervical cancer, ovary cancer, lymfoma's, melanomia and leukemia.

22. Method according to claim 20, wherein the matrine component is administered in an average daily amount of 0.5-10 μmole per kg of bodyweight.

23. Method according to claim 20, wherein dictamnine is administered in an average daily amount of 4-10 namomoles per kg of bodyweight.

24. Method according to claim 20, wherein the method comprises co-administering a triterpenoid component selected from the group consisting of sapogenins, limonoids, triterpenoids with an oleanane skeleton, triterpenoids with a lupane skeleton, precursors of these triterpenoid components and mixtures thereof, in an average daily amount of at least 40 nanomoles per kg of bodyweight, preferably of 80-4800 nanomoles per kg of bodyweight.

25. Method according to claim 20, wherein the method comprises the co-administration of aurapten in an average daily amount of 0.005 to 20 mg per kg of bodyweight, preferably of 0.001 to 10 mg per kg of bodyweight.

26. A method of treating or preventing Helicobacter pylori infections in a mammal, said method comprising administering to the mammal a composition containing a matrine component and dictamnine, in an amount effective to deliver an average daily amount of at least 0.2 μmole matrine component per kg of bodyweight and an average daily amount of at least 2 nanomole dictamnine per kg of bodyweight.

27. A method of treating or preventing chronic inflammatory conditions in a mammal, said method comprising administering to the mammal a composition containing a matrine component and dictamnine, in an amount effective to deliver an average daily amount of at least 0.2 μmole matrine component per kg of bodyweight and an average daily amount of at least 2 nanomole dictamnine per kg of bodyweight.

28. A method of treating or preventing cardiovascular diseases in a mammal, said method comprising administering to the mammal a composition containing a matrine component and dictamnine, in an amount effective to deliver an average daily amount of at least 0.2 μmole matrine component per kg of bodyweight and an average daily amount of at least 2 nanomole dictamnine per kg of bodyweight.

29. A method of treating or preventing cerebral vascular diseases in a mammal, said method comprising administering to the mammal a composition containing a matrine component and dictamnine, in an amount effective to deliver an average daily amount of at least 0.2 μmole matrine component per kg of bodyweight and an average daily amount of at least 2 nanomole dictamnine per kg of bodyweight.

30. A unit dosage of a nutritional composition or a pharmaceutical unit dosage selected from the group consisting of a tablet, a lozenge, an elixir, a liquid, a powder or a suppository, comprising at least 5 μmole of a matrine component and at least 0.05 μmole of dictamnine.

31. Unit dosage according to claim 30, comprising at least 15 μmole of a matrine component and at least 0.15 μmole of dictamnine.

32. Unit dosage according to claim 30, additionally comprising at least 2 mg of aurapten.

33. Unit dosage according to claim 30, wherein the unit dosage comprises plant material derived from a plant belonging to the Sophora species, preferably Sophora flavescens or Sophora subprostata.

34. Unit dosage according to claim 30, wherein the unit dosage comprises plant material derived from a plant belonging to the species Dictamnus.

35. A pharmaceutical unit dosage according to claim 30, wherein the remainder largely consists of pharmaceutically acceptable excipient.
